(19) United States
(12) Patent Application Publication
(10) Pub. No.: US 2007/0259895 A1
(43) Pub. Date: Nov. 8, 2007

(54) MGluR5 modulators VI

(75) Inventors: Methvin Isaac, Brampton (CA); Abdelmalik Slassi, Mississauga (CA); Louise Edwards, Mississauga (CA); Tao Xin, Woodbridge (CA); Tomislav Stefanac, Burlington (CA); Peter Dove, Toronto (CA); Mats Nagard, Molndal (SE)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB, Sodertalje (SE); NPS PHARMACEUTICALS, INC., Salt Lake City, UT (US)

(21) Appl. No.: 11/790,424

(22) Filed: Apr. 25, 2007

Related U.S. Application Data
(60) Provisional application No. 60/797,664, filed on May 5, 2006.

(51) Int. Cl. A61K 31/519; C07D 487/04

(52) U.S. Cl. 514/259.31; 544/255

(57) ABSTRACT

The present invention is directed to novel compounds, to a process for their preparation, their use in therapy and pharmaceutical compositions comprising the novel compounds.

### FIELD OF THE INVENTION

The present invention is directed to novel compounds, their use in therapy and pharmaceutical compositions comprising said novel compounds.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

### Neurological, Psychiatric and Pain Disorders

Attempts at elucidating the physiological roles of Group I mGluRs suggest that activation of these receptors elicits neuronal excitation. Various studies have demonstrated that Group I mGluR agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci, 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastrointestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current—sub-units that are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

Generally, compounds of the present invention have low activity against the hERG-encoded potassium channel. In this regard, low activity against hERG in vitro is indicative of low activity in vivo.

It is also desirable for drugs to possess good metabolic stability in order to enhance drug efficacy. Stability against human microsomal metabolism in vitro is indicative of stability towards metabolism in vivo.

Because of their physiological and pathophysiological significance, there is a need for new potent mGluR agonists and antagonists that display a high selectivity for mGluR subtypes, particularly the Group I receptor subtype, most particularly the mGluR5.

The object of the present invention is to provide compounds exhibiting an activity at metabotropic glutamate receptors (mGluRs), especially at the mGluR5 receptor. In particular, the compounds according to the present invention are predominantly peripherally acting, i.e. have a limited ability of passing the blood-brain barrier.

### DESCRIPTION OF THE INVENTION

The present invention relates to a compound of formula I:

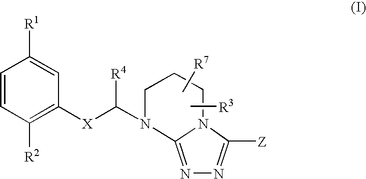

wherein

### R¹ is methyl, halogen or cyano;

### R² is hydrogen or fluoro;

### R³ is hydrogen, fluoro or C₁-C₃ alkyl;

### R⁴ is hydrogen or C₁-C₃ alkyl;

### X is

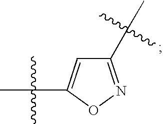

and Z is

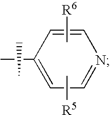

### R⁵ is hydrogen, C₁-C₃ alkyl, C₁-C₃ haloalkyl, C₁-C₃ alkoxy; or C₁-C₃ haloalkoxy;

### R⁶ is hydrogen, C₁-C₃ alkyl, C₁-C₃ haloalkyl, or C₁-C₃ haloalkoxy;

### R⁷ is hydrogen, fluoro or C₁-C₃ alkyl;

as well as pharmaceutically acceptable salts, hydrates, isoforms, tautomers and/or enantiomers thereof.

In one embodiment R¹ is halogen or cyano.

In a further embodiment, R¹ is chloro. In a further embodiment, R¹ is cyano.

In a further embodiment, R² is hydrogen.

In a further embodiment, R³ is hydrogen or fluoro.

In a further embodiment, R⁴ is hydrogen or methyl.

In a further embodiment, R⁵ is hydrogen, C₁-C₂ alkyl or C₁-C₂ alkoxy.

In a further embodiment, R⁶ is hydrogen, C₁-C₂ alkyl or C₁-C₂ alkoxy.

In a further embodiment, R⁷ is C₁-C₂ alkyl or C₁-C₂ alkoxy.

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGluR5 receptors, comprising treating a cell containing said receptor with an effective amount of the compound according to formula I.

The compounds of the present invention are useful in therapy, in particular for the treatment of neurological, psychiatric, pain, and gastrointestinal disorders.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl, acetic acid or a methanesulfonic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The general terms used in the definition of formula I have the following meanings:

Halogen as used herein is selected from chlorine, fluorine, bromine or iodine.

C₁-C₃ alkyl is a straight or branched alkyl group, having from 1 to 3 carbon atoms, for example methyl, ethyl, n-propyl or isopropyl.

C₁-C₃ alkoxy is an alkoxy group having 1 to 3 carbon atoms, for example methoxy, ethoxy, isopropoxy or n-propoxy.

C₁-C₃ haloalkoxy is an alkoxy group having 1 to 3 carbon atoms, for example methoxy, ethoxy or n-propoxy wherein at least one of the carbon atoms is substituted by a halogen atom.

All chemical names were generated using a software known as AutoNom accessed through ISIS draw.

In formula I above, X may be present in any of the two possible orientations.

### Pharmaceutical Composition

The compounds of the present invention may be formulated into conventional pharmaceutical compositions comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, or from about 0.10% w to 50% w, of a compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

The compounds according to the present invention are useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of formula I, as defined hereinbefore, for use in therapy.

The invention relates to compounds of formula I, as defined hereinbefore, for use in treatment of mGluR5-mediated disorders.

The invention relates to compounds of formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or billiary colic, menstruation, migraine and gout.

The invention relates to compounds of formula I as defined hereinbefore, for use in treatment of stroke, head trauma, anoxic and ischemic injuries, hypoglycemia, cardiovascular diseases and epilepsy.

The present invention relates also to the use of a compound of formula I as defined hereinbefore, in the manufacture of a medicament for the treatment of mGluR Group I receptor-mediated disorders and any disorder listed above.

One embodiment of the invention relates to the use of a compound according to formula I in the treatment of gastrointestinal disorders.

Another embodiment of the invention relates to the use of a formula I compound for the manufacture of a medicament for inhibition of transient lower esophageal sphincter relaxations, for the treatment of GERD, for the prevention of gastroesophageal reflux, for the treatment regurgitation, for treatment of asthma, for treatment of laryngitis, for treatment of lung disease, for the management of failure to thrive, for the treatment of irritable bowel disease (IBS) and for the treatment of functional dyspepsia (FD).

Another embodiment of the present invention relates to the use of a compound of formula I for treatment of overactive bladder or urinary incontinence.

The wording “TLESR”, transient lower esophageal sphincter relaxations, is herein defined in accordance with Mittal, R. K, Holloway, R. H., Penagini, R, Blackshaw, L. A., Dent, J, 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000, Diagnosis of reflux disease. Baillière's Clin. Gastroenterol. 14, pp. 759-774.

The compounds of formula I above are useful for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive) and cravings (for drugs, tobacco, alcohol, any appetizing macronutrients or non-essential food items).

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

One embodiment of the present invention is a combination of a compound of formula I and an acid secretion inhibiting agent. A “combination” according to the invention may be present as a “fix combination” or as a “kit of parts combination”. A “fix combination” is defined as a combination wherein the (i) at least one acid secretion inhibiting agent; and (ii) at least one compound of formula I are present in one unit. A “kit of parts combination” is defined as a combination wherein the (i) at least one acid secretion inhibiting agent; and (ii) at least one compound of formula I are present in more than one unit. The components of the “kit of parts combination” may be administered simultaneously, sequentially or separately. The molar ratio of the acid secretion inhibiting agent to the compound of formula I used according to the invention in within the range of from 1:100 to 100:1, such as from 1:50 to 50:1 or from 1:20 to 20:1 or from 1:10 to 10:1. The two drugs may be administered separately in the same ratio. Examples of acid secretion inhibiting agents are H2 blocking agents, such as cimetidine, ranitidine; as well as proton pump inhibitors such as pyridinylmethylsulfinyl benzimidazoles such as omeprazole, esomeprazole, lansoprazole, pantoprazole, rabeprazole or related substances such as leminoprazole.

### Non-Medical Use

In addition to their use in therapeutic medicine, the compounds of formula I, as well as salts and hydrates of such compounds, are useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutic agents.

### Methods of Preparation

Another aspect of the present invention provides a process for preparing a compound of formula I or salt thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, 1999. Throughout the following description of such processes it is to be understood that cross-couplings can be performed in a manner that will be readily understood by one skilled in the art of organic synthesis. Conventional procedures for cross-coupling are described, for example, in “Organometallics in Synthesis”, M. Schlosser (Ed.), John Wiley and Sons (2001).

### Abbreviations:

atm Atmosphere
aq. Aqueous
BINAP 2,2′-bis(diphenylphosphino)-1,1′-binaphthyl
Boc tert-butoxycarbonyl
CDI N,N′-Carbonyldiimidazole
DCC N,N-Dicyclohexylcarbodiimide
DCM Dichloromethane
DBU Diaza(1,3)bicyclo[5.4.0]undecane
DEA N,N-Diisopropyl ethylamine
DIBAL-H Diisobutylaluminium hydride
DIC N,N′-Diisopropylcarbodiimide
DMAP N,N-Dimethyl-4-aminopyridine
DMF Dimethylformamide
DMSO Dimethylsulfoxide
DPPF Diphenylphosphinoferrocene
EA Ethyl acetate
EDCl N-[3-(dimethylamino)propyl]-N′-ethylcarbodiimide hydrochloride
EDC 1-Ethyl-3-(3-dimethylaminopropyl)carbodiimide
Et₂O Diethyl-ether
EtOAc Ethyl acetate
EtOH Ethanol
EtI Iodoethane
Et Ethyl
Fmoc 9-fluorenylmethyloxycarbonyl
h hour(s)
HetAr Heteroaryl
HOBt N-Hydroxybenzotriazole
HBTU O-(Benzotriazol-1-yl)-N,N,N′,N′-tetramethyluronium hexafluorophosphate
HPLC High performance liquid chromatography
LAH Lithium aluminium hydride
LCMS HPLC mass spec
MCPBA m-Chlorbenzoic acid
MeCN Acetonitrile
MeOH Methanol
min Minutes
MeI Iodomethane
MeMgCl Methyl magnesium chloride
Me Methyl
n-BuLi I-Butyllithium
NaOAc Sodium acetate
NMR Nuclear magnetic resonance
NMP N-Methyl pyrrolidinone
nBuLi 1-Butyl lithium
o.n. Over night
RT, rt, r.t. Room temperature
TEA Triethylamine
THF Tetrahydrofurane
nBu normal Butyl
OMs Mesylate or methane sulfonate ester
OTs Tosylate, toluene sulfonate or 4-methylbenzene sulfonate ester
PCC Pyridinium chlorochromate
PPTS Pyridinium p-toluenesulfonate
TBAF Tetrabutylammonium fluoride
pTsOH p-Toluenesulfonic acid
SPE Solid phase extraction (usually containing silica gel for mini-chromatography)
sat. Saturated

### Formation of Isoxazole Precursor of Compounds of Formula I

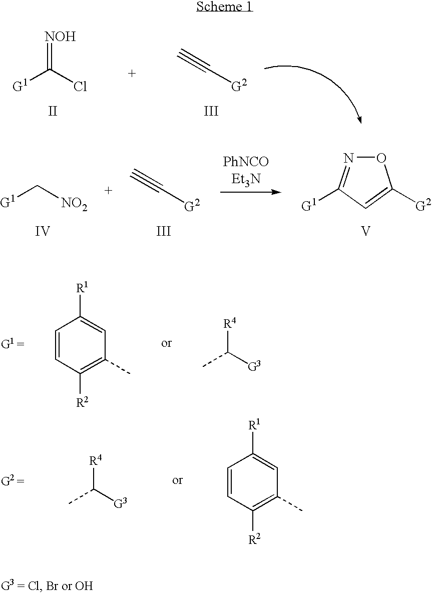

A compound of formula I, may be prepared by a 1,3-dipolar cycloaddition between compounds of formula II and III under basic conditions using a suitable base such as sodium bicarbonate or triethylamine at suitable temperatures (0° C.-100° C.) in solvents such as toluene. Synthesis of compounds of type II has previously been described in the literature, e.g. Kim, Jae Nyoung; Ryu, Eung K; J. Org. Chem. (1992), 57, 6649-50. 1,3-Dipolar cycloaddition with acetylenes of type III can also be effected using substituted nitromethanes of type IV via activation with an electrophilic reagent such as PhNCO in the presence of a base such as triethylamine at elevated temperatures (50-100° C.). Li, C—S.; Lacasse, E.; Tetrahedron Lett. (2002) 43; 3565-3568. Several compounds of type III are commercially available, or may be synthesized by standard methods as known by one skilled in the art.

Alternatively, compounds of formula I (X is isoxazole) which are available from a Claisen condensation of a methyl ketone VI and an ester using basic conditions using such bases as sodium hydride or potassium tert-butoxide, may yield compounds of formula VIII via condensation and subsequent cyclization using hydroxylamine, for example in the form of the hydrochloric acid salt, at elevated temperatures (60-120° C.).

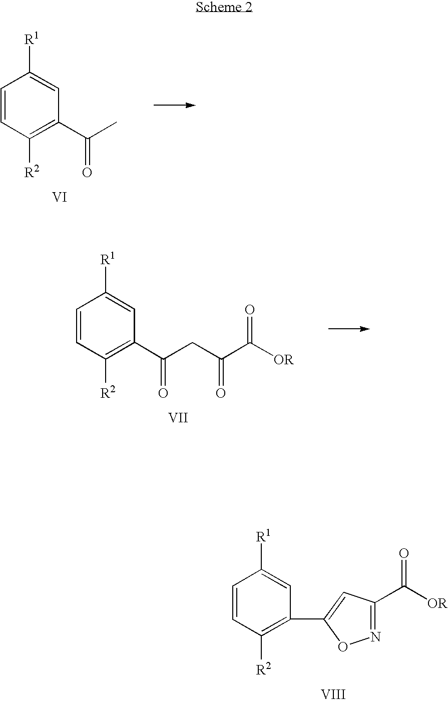

It is understood that for both methods subsequent functional group transformations may be necessary. In the case of an ester group, these transformations may include, but is not limited to either of following three procedures: a) Complete reduction using a suitable reducing agent such as LAH in solvents such as THF. b) Partial reduction using a suitable selective reducing agent such as DIBAL followed by addition of an alkylmetal reagent. c) Addition of an alkylmetal reagent such as an alkyl magnesium halide in solvents such as toluene or THF, followed by reduction with for example sodium borohydride in methanol. The compounds, and the corresponding intermediates throughout the non-limiting synthetic paths for which preparations are given below, are useful for further preparation of compounds of formula I or may represent the same. Other starting materials are either commercially available or can be prepared via methods described in the literature.

### Preparation of Amino[1,2,4]triazole intermediates

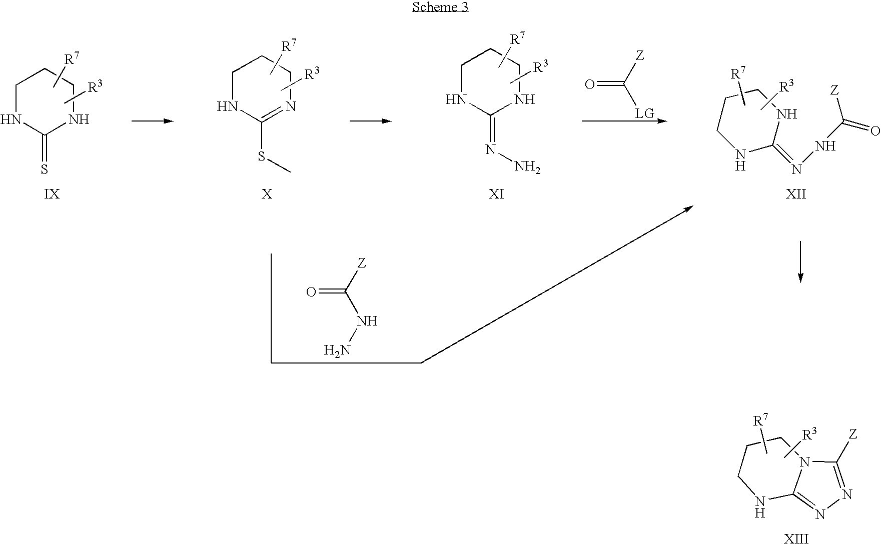

With reference to Scheme 3, amino[1,2,4]triazoles XIII are obtained by treating carbono-hydrazonic diamides XI with a proper acylating agent carrying a leaving group (LG) in suitable solvent such as THF, pyridine or DMF at −20-100° C. The reaction initially leads to an open intermediate XII that either forms a triazole ring spontaneously, or can be made to do so by heating at 50-200° C. in for example pyridine or DMF. The leaving group (LG) may be chloro or any other suitable leaving group as for example generated by in situ treatment of the corresponding acid (LG is OH) with standard activating reagents as described herein below. Carbonohydrazonic diamides XI may be generated from isothioureas IX, in which the S-alkyl (for example S-Me as shown in scheme 4) moiety acts as a leaving group upon treatment with hydrazine in solvents such as pyridine, methanol, ethanol, 2-propanol, THF, DMSO or the like at −20 to 180° C. The open intermediate XII can also be directly generated by treatment of isothioureas with acylhydrazines under the same conditions as described for the reaction with hydrazine. Isothioureas are obtained by S-alkylation of the corresponding thioureas with for example MeI or EtI in acetone, EtOH, THF, DCM or the like at −100-100° C.

### Functional Group Transformations of Compounds of Formula VIII

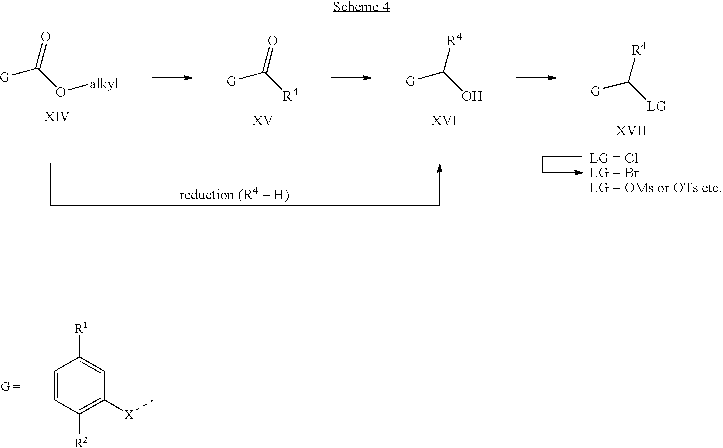

With reference to Scheme 4, alcohols XVI may for example be converted by standard methods to the corresponding halides XVII (LG=Cl, Br etc.) by the use of for example triphenylphosphine in combination with either iodine, N-bromosuccinimide or N-chlorosuccinimide, or alternatively by treatment with tribromo phosphine or thionylchloride. In a similar fashion alcohols XVI may be transformed to other leaving groups such as mesylates or tosylates by employing the appropriate sulfonyl halide or sulfonyl anhydride in the presence of a non-nucleophilic base together with the alcohol to obtain the corresponding sulfonates. Chlorides or sulphonates can be converted to the corresponding bromides or iodides by treatment with bromide salts, for example LiBr, or iodide salts. Further standard methods to obtain alcohols XVI include the reduction of the corresponding carbonyl containing groups as in XIV and XV (such as methyl or ethyl esters, aldehydes (R⁴ is H) or ketones (R⁴ is not H), by employing common reducing agents such as boranes, lithium borohydride, lithium aluminumhydride, or hydrogen in the presence of a transition metal catalyst such as complexes of for example ruthenium or iridium, or alternatively palladium on charcoal.

### General Syntheses of Compounds of Formula I

The subsequent described non-limiting methods of preparation of final compounds are illustrated and exemplified by drawings in which the generic groups, or other structural elements of the intermediates correspond to those of formula I. It is to be understood that an intermediate containing any other generic group or structural element than those of formula I can be used in the exemplified reactions, provided that this group or element does not hinder the reaction and that it can be chemically converted to the corresponding group or element of formula I at a later stage which is known to the one skilled in the art.

### By Connection to Nucleophilic Triazole Nitrogen

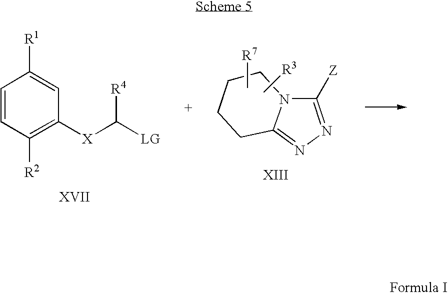

With reference to scheme 5, compounds of formula I can be prepared by bond formation through nucleophilic replacement of a leaving group (LG) in which the triazole exocyclic NH moiety is acting as nucleophile. The nitrogen atom of the triazole in its anionic form, generated by treatment of the corresponding protonated neutral atom with bases in suitable solvents such as LDA or nBuLi in THF, diethylether or toluene, or NaH or NaOtBu in for example DMF, or K₂CO₃ in acetonitrile or ketones such as 2-butanone at a temperature from −100-150° C. The LG is preferably chloro, bromo, OMs and OTs. The nucleophilic reaction may also be undertaken in a stereoselective manner by employing enantiomerically pure or enriched starting materials in which the leaving group LG is attached to the stereocenter. Optionally, catalytic or stoichiometric amounts of an alkali metal iodide, such as LiI, can be present in the reaction to facilitate the same through in situ displacement of the leaving group to iodo.

Embodiments of the present invention will now be illustrated by the following non-limiting examples.

### General Methods

All starting materials are commercially available or earlier described in the literature. The ¹H and ¹³C NMR spectra were recorded on one of a Bruker 300 at 300 MHz Bruker, DPX400 at 400 MHz or Varian +400 spectrometer at 100 MHz, using TMS or the residual solvent signal as reference. NMR measurements were made on the delta scale (δ). Mass spectra were recorded on a QTOF Global Micromass or a Waters LCMS consisting of an Alliance 2795 (LC) and a ZQ single quadropole mass spectrometer. The mass spectrometer was equipped with an electrospray ion source operated in a positive or negative ion mode. The ion spray voltage was ±3 kV and the mass spectrometer was scanned from m/z 100-700 with a scan time of 0.8 s. Column: X-Terra MS, Waters, C8, 2.1×50 mm, 3.5 μM and the column temperature was set to 40° C. A linear gradient was applied, run at 0% to 100% acetonitrile in 4 minutes, flow rate 0.3 mL/min. Mobile phase: acetonitrile/10 mM ammonium acetate in 5% acetonitrile in MilliQ Water. Preparative chromatography was run on a Gilson autopreparative HPLC with a diode array detector. Column: XTerra MS C8, 19×300 mm, 7 μm. Gradient with acetonitrile/0.1 M ammonium acetate in 5% acetonitrile in MilliQ Water, generally run from 20% to 60% acetonitrile, in 13 min. Flowrate: 20 mL/min. MS-triggered prep-LC was run on a Waters autopurification LC-MS system with a diode array detector and a ZQ mass detector. Column: XTerra MS C8, 19×100 mm, 5 μm. Gradient with acetonitrile/0.1 M ammonium acetate in 5% acetonitrile in MilliQ Water, run from 0% to 100% acetonitrile, in 10 min. Flowrate: 20 mL min. In some cases purification by a chromatotron was performed on rotating silica gel/gypsum (Merck, 60 PF-254 with calcium sulphate) coated glass sheets, with coating layer of 2 mm using a TC Research 7924T chromatotron. Alternatively Chem Elut Extraction Column (Varian, cat #1219-8002) and Mega BE-SI (Bond Elut Silica) SPE Columns (Varian, cat #12256018; 12256026; 12256034) were used during purification of the products.

The microwave heating was performed in a Smith Synthesizer Single-mode microwave cavity producing continuous irradiation at 2450 MHz (Personal Chemistry AB, Uppsala, Sweden).

### EXAMPLES

The invention will now be illustrated by the following non-limiting examples.

### Example 1

### 4-(3-Chloro-phenyl)-2,4-dioxo-butyric acid ethyl ester

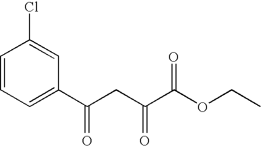

Sodium hydride (60% oil dispersion, 1.24 g, 31.1 mmol) was added in portions to a solution of 3-chloroacetophenone (4.0 g, 25.9 mmol) and diethyl oxalate (4.54 g, 31.1 mmol) in DMF (32 mL) at 0° C. The mixture stirred at room temperature for 1 hour and was then heated at 80° C. for a half an hour. After cooling, the mixture was treated with 3 M HCl and then diluted with ethyl acetate. The organic layer washed three times with water and saturated brine, dried over anhydrous sodium sulfate, filtered and concentrated. The resulting residue was then purified by flash column chromatography on silica using 0-10% ethyl acetate in hexanes to afford of the title compound (4.43 g, 67%, yellow solid).

¹H NMR (300 MHz, CDCl₃): δ 15.12 (br s, 1H), 7.98 (s, 1H), 7.88 (d, 1H), 7.58 (d, 1H), 7.47 (t, 1H), 7.05 (s, 1H), 4.39 (m, 2H), 1.41 (m, 3H).

### Example 2

### 5-(3-Chloro-phenyl)-isoxazole-3-carboxylic acid ethyl ester and 5-(3-Chloro-phenyl)-isoxazole-3-carboxylic acid methyl ester

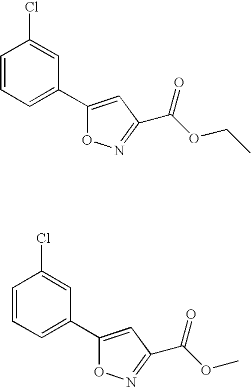

A solution of the title compound from Example 1 (3.00 g, 11.8 mmol) and hydroxylamine hydrochloride (2.46 g, 35.4 mmol) in methanol (60 mL) was heated at 80° C. for 4 hours. After cooling, the mixture was filtered and washed with cold methanol to afford 2.0 g of the title compound (yield 71%) as a white solid. Mixture of both methyl and ethyl ester (predominantly methyl).

¹H NMR (300 MHz, CDCl₃): δ 7.82 (s, 1H), 7.72 (m, 1H), 7.47 (m, 2H), 4.03 (s, 3H).

### Example 3

### [5-(3-Chloro-phenyl)-isoxazol-3-yl]-methanol

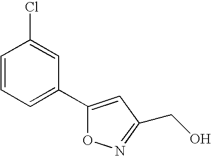

Lithium aluminum hydride (320 mg, 8.4 mmol) was slowly added to a solution of the title compounds of Example 2 (2.0 g, 8.4 mmol) in THF (100 mL) at room temperature. After 1 hour, the reaction mixture was quenched with water and then extracted with ethyl acetate. The organic layer washed with water and saturated brine, dried over anhydrous sodium sulfate, filtered, and concentrated. The resulting residue was then purified by flash column chromatography using 15-40% ethyl acetate in hexane to give 1.32 g of the title compound (75% yield) as a yellow solid.

¹H NMR (300 MHz, CDCl₃): δ 7.78 (s, 1H), 7.68 (m, 1H), 7.43 (m, 2H), 6.63 (s, 1H), 4.84 (d, 2H), 2.23 (t, 1H).

### Example 4

### Methanesulfonic acid 5-(3-chloro-phenyl)-isoxazol-3-ylmethyl ester

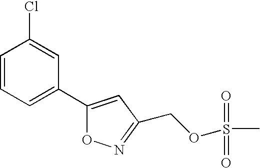

Triethyl amine (965 mg, 9.5 mmol) and methanesulfonyl chloride (820 mg, 7.2 mmol) were added to a solution of the title compound of Example 3 (1.0 g, 4.8 mmol) in dichloromethane (50 mL) at 0° C. After 1 hour, the reaction mixture was quenched with cold saturated sodium bicarbonate and then the organic layer washed with saturated brine, dried over anhydrous sodium sulfate, filtered, and concentrated to afford 1.4 g (100% yield) of the title compound as a light brown solid.

¹H NMR (300 MHz, CDCl₃): δ 7.80 (s, 1H), 7.70 (m, 1H), 7.45 (m, 2H), 6.73 (s, 1H), 5.37 (s, 2H), 3.16 (s, 3H).

### Example 5

### 1-[5-(3-Chloro-phenyl)-isoxazol-3-yl]-ethanone

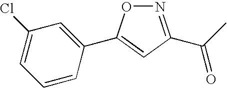

In a screw cap vial equipped with stir bar added methyl magnesium iodide (3 M in diethyl ether) (0.79 mL, 2.38 mmol), toluene (1 mL), tetrahydrofuran (0.39 mL, 4.77 mmol) and triethylamine (1 mL, 7.15 mmol). Cooled the solution down to 0° C. and to it added solution of the title compound of Example 2 (300 mg, 1.19 mmol) in toluene (5 mL). Left the resulting mixture stirring at 0° C. for 5 h. The reaction mixture was quenched with 1 M hydrochloric acid (aqueous, 6.5 mL, 6.5 mmol), diluted with toluene (35 mL), sequentially washed with water (50 mL), saturated sodium bicarbonate (aqueous, 30 mL), water (50 mL) and brine (30 mL). The organic phase was concentrated, in-vacuo. The isolated residue was dissolved in methanol (8 mL) and 20% potassium hydroxide (aqueous, 1 mL). The mixture was stirred at 45° C. for 30 minutes. At this point the mixture was concentrated, in-vacuo. The isolated residue was dissolved in toluene (60 mL), sequentially washed with water (50 mL), saturated sodium bicarbonate (aqueous, 50 mL) and water (50 mL). The organic phase was concentrated in-vacuo. The crude residue was purified on silica gel using 2% ethyl acetate in hexanes to isolate the title compound as a white solid (156 mg, 60% yield).

¹H NMR (300 MHz, CDCl₃): δ 7.77 (m, 1H), 7.66 (m, 1H), 7.42 (m, 2H), 6.90 (s, I H), 2.69 (s, 3H).

### Example 6

### Methanesulfonic acid 1-[5-(3-chloro-phenyl)-isoxazol-3-yl]-ethyl ester

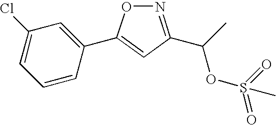

### Step A, 1-[5-(3-Chloro-phenyl)-isoxazol-3-yl]-ethanol

In a screw cap vial equipped with stir bar added the title compound of Example 5 (100 mg, 0.45 mmol), sodium borohydride (34 mg, 0.90 mmol) and methanol (3 mL). Left the resulting mixture stirring at room temperature for 3 h. The reaction was quenched with water (30 mL) and brine (30 mL), extracted with dichloromethane (three times 30 mL). The combined organic phase was dried (sodium sulfate), filtered and concentrated, in vacuo to isolate the subtitle compound as a white solid (110 mg).

¹H NMR (300 MHz, CDCl₃): δ 7.69 (m, 1H), 7.59 (m, 1H), 7.37 (m, 2H), 6.59 (s, 1H), 5.07 (q, 1H), 3.45 (bs, 1H), 1.58 (d, 3H).

### Step B

In a screw cap vial equipped with stir bar added the subtitle compound from Step 6A (110 mg, 0.49 mmol), dichloromethane (3 mL) and triethylamine (0.34 mL, 2.46 mmol). Cooled the mixture down to 0° C. and to it added methane sulfonyl chloride (0.080 mL, 0.98 mmol). Left the reaction mixture stirring at room temperature for 30 minutes. The reaction was quenched with saturated sodium bicarbonate (aqueous, 40 mL) and extracted with dichloromethane (3 times 30 mL). Combined organic phase washed with brine (40 mL), dried (sodium sulfate), filtered and concentrated, in-vacuo to isolate the subtitle compound as brown oil which was used directly in the next step.

### Example 7

### 3-[3-(1-hydroxyethyl)isoxazol-5-yl]benzonitrile

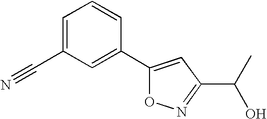

### Step A: 4-(3-Iodo-phenyl)-2,4-dioxo-butyric acid methyl ester

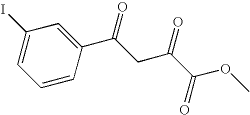

Sodium hydride (60% oil dispersion, 4.9 g, 123 mmol) was added in portions to a solution of 3-iodoacetophenone (25.18 g, 102.3 mmol) and dimethyl oxalate (14.5 g, 123 mmol) in DMF (125 mL) at 0° C. The mixture stirred at room temperature for 1 hour and was then heated at 115° C. for 1 h. After cooling, the mixture was treated with 3 M HCl and then diluted with ethyl acetate. The organic layer washed three times with water and saturated brine, dried over anhydrous sodium sulfate, filtered and concentrated. Chromatography on silica gel, 0-10% ethyl acetate in hexanes, afforded 24.2 g of the subtitle compound (71.3% yield) as a yellow solid which was used directly in the next step.

### Step B: 5-(3-Iodo-phenyl)-isoxazole-3-carboxylic acid methyl ester

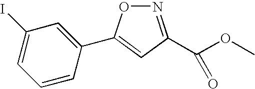

A solution of the subtitle compound of Step 7A (33.9 g, 102 mmol) and hydroxylamine hydrochloride (21.3 g, 306 mmol) in methanol (450 mL) was heated at reflux for 4 hours. After cooling, the mixture was filtered and washed with cold methanol to afford the subtitle compound (24.1 g, 72%, brown solid).

¹H NMR (300 MHz, CDCl₃): δ 8.18 (m, 1H), 7.82 (t, 2H), 7.26 (t, 1H), 6.97 (s, 1H), 4.03 (s, 3H).

### Step C: [5-(3-Iodophenyl)isoxazol-3-yl]methanol

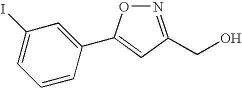

DIBAL (55.8 mL, 1.5 M in toluene, 83.7 mmol) was slowly added to the subtitle compound of Step 7B (12 g, 36.5 mmol) in toluene (60 mL) and THF (60 mL) at −78° C. The resulting mixture was stirred at −78° C. overnight, then allowed to warm slowly to RT. The reaction was quenched with a mixture of ice and saturated ammonium chloride (aqueous). The product was extracted with ethyl acetate, and the organic layer washed with brine, dried over sodium sulfate and concentrated in vacuo to give the title compound (off-white solid, 10.5 g, 95.6%).

¹H NMR (300 MHz, CDCl₃): δ 8.12 (m, 1H), 7.76 (ddm, 2H), 7.21 (t, 1H), 6.62 (s, 1H), 4.83 (s, 2H), 2.45 (br s, 1H).

### Step D: 5-(3-Iodophenyl)isoxazole-3-carbaldehyde

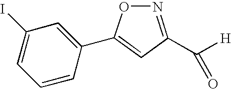

The crude reaction mixture from Step 7C (8.5 g, 28.2 mmol) and PCC (9.13 g, 42.3 mmol) in dichloromethane (150 mL) was stirred at room temperature overnight. The mixture was diluted with 15% ethyl acetate in hexanes and passed thorough a short plug of silica gel, eluting with additional 15% ethyl acetate in hexanes. The eluent was concentrated in vacuo to give the subtitle compound as a pale yellow solid, 7.0 g (83% yield).

¹H NMR (300 MHz, CDCl₃): δ 10.21 (s, 1H), 8.19 (m, 1H), 7.83 (ddm, 2H), 7.27 (m, 1H), 6.93 (s, 1H).

### Step E: 1-[5-(3-Iodo-phenyl)-isoxazol-3-yl]-ethanol

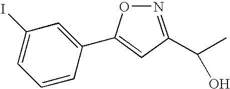

Methyl magnesium iodide (33 mL, 3 M in diethyl ether, 99 mmol) was added to a cold (0° C.) solution of the subtitle compound from Step 7D (7.5 g, 25 mmol) in THF (100 mL). The reaction mixture was stirred at 0° C. for 1 h and quenched with saturated ammonium chloride. The product was extracted with ethyl acetate, and the organic layer washed with brine, dried over a mixture of sodium sulfate and silica gel. The filtrate was concentrated in vacuo and chromatography (silica, 15-50% ethyl acetate in hexanes) gave the crude iodo-isoxazole-alcohol as a pale yellow oil, 6.5 g, contaminated with ˜33% 1-(5-phenylisoxazol-3-yl)ethanol).

### Step F: 3-[1-(tert-Butyl-dimethyl-silanyloxy)-ethyl]-5-(3-iodo-phenyl)-isoxazole

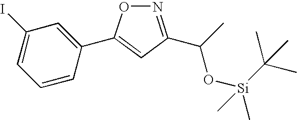

Tert-butyldimethylchlorosilane (2.5 g, 2.3 mmol) was added to a solution of the crude material of Step 7E (4.9 g, 15.5 mmol) and DBU (2.53 g, 2.13 mmol) in dichloromethane (60 mL) and the reaction was stirred at RT for 3 h. Tert-butyldimethylchlorosilane (2.5 g, 2.3 mmol) and DBU (2.53 g, 2.13 mmol) were added and stirring was continued for 15 min until TLC indicated the alcohol was consumed. The product was partitioned between saturated ammonium chloride and dichloromethane, and the organic layer was dried and concentrated in vacuo to give the subtitle compound as a pale yellow solid)(8.4 g crude).

### Step G: 3-{3-[1-(tert-Butyl-dimethyl-silanyloxy)-ethyl]-isoxazol-5-yl}-benzonitrile

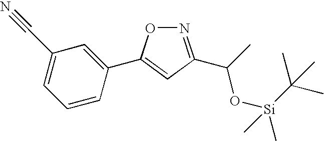

A mixture of the crude product from Step 7F, zinc cyamide (1.6 g, 13.7 mmol), tetrakis(triphenylphosphine)palladium(0) (1.58 g, 1.37 mmol) in DMF (100 mL) was stirred at 82° C. for 10 min. The mixture was diluted with ethyl acetate and filtered through celite. The filtrate was concentrated in vacuo and diluted with dichloromethane. The solution washed with water, dried over sodium sulfate and filtered. Chromatography (preadsorbed on silica, 1-5% ethyl acetate in hexane) gave the subtitle compound as an off-white solid (3.83 g, 46.5% over 3 steps).

¹H NMR (300 MHz, CDCl₃): δ 8.07 (m, 1H), 8.04 (dm, 1H), 7.73 (dm, 1H), 7.62 (t, 1H), 6.66 (s, 1H), 5.09 (q, 1H), 1.54 (d, 3H), 0.93 (s, 9H), 0.13 (s, 3H), 0.06 (s, 3H).

### Step H: 3-[3-(1-Hydroxy-ethyl)-isoxazol-5-yl]-benzonitrile

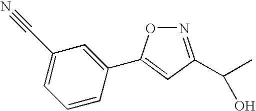

TBAF (20 mL, 1 M in THF, 20 mmol) was added to a solution of the pure cyano-isoxazole-silyl ether (3.83 g, 11.7 mmol) in THF (40 mL) at 0° C. and the mixture was stirred overnight at RT. The product was partitioned between dichloromethane and water. The organic layer washed with brine and dried over magnesium sulfate. Silica gel was added and the mixture was passed through a plug of silica gel using 50% ethyl acetate in hexanes. The eluent was concentrated in vacuo and the residue was triturated with hexanes to give the title compound as an off-white solid, 2.5 g (100% yield).

¹H NMR (300 MHz, CDCl₃): δ 8.07 (m, 1H), 8.03 (dm, 1H), 7.75 (dm, 1H), 7.62 (t, 1H), 6.7 (s, 1H), 5.13 (q, 1H), 1.64 (d, 3H).

### Example 8

### 1-[5-(3-Cyanophenyl)isoxazol-3-yl]ethyl methanesulfonate

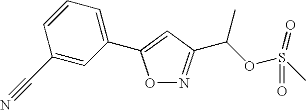

Methanesulfonyl chloride (1.5 mmol) and triethylamine (2 mmol) were added to a solution of the title compound of Example 7 (1 mmol) in dichloromethane (10-15 mL) at 0° C. The reaction mixture was stirred at 0° C. for 30 minutes, then washed with cold saturated sodium bicarbonate. The organic layer washed with brine, dried with sodium sulfate and concentrated in vacuo to give 3.65 g of the title compound as an off-white solid, which was used without further purification (100% yield).

¹H NMR (300 MHz, CDCl₃): δ 8.09 (m, 1H), 8.04 (dm, 1H), 7.77 (dm, 1H), 7.65 (t, 1H), 6.77 (s, 1H), 5.94 (q, 1H), 3.08 (s, 3H), 1.85 (d, 3H).

### Example 9

### General Procedure for the Formation of Cyclic Triazole Intermediates

The acid chloride was added to a vial followed by pyridine (˜0.5 mL/mmol). The hydrazine (1 equivalent) was then added to the solution and refluxed at 130° C. over night. The solution was basified using potassium carbonate and aqueous workup was then performed using EtOAc, water, and Brine. The organic layer was dried over anhydrous sodium sulfate, filtered and concentrated. An SPE/Flash column was run using a 10-20% MeOH:EtOAc solvent system. The eluting fractions were collected and concentrated. The following table depicts the aminotriazoles formed.

### Example 9.1

### 3-Pyridin-4-yl-5,6,7,8-tetrahydro-[1,2,4]triazolo[4,3-a]pyrimidine

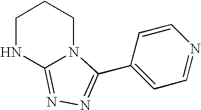

A solution of 750 mg (3.1 mmol) (1,4,5,6-tetrahydro-pyrimidin-2-yl)-hydrazine hydroiodide (ref. Krezel, Izabella; Pharmazie; EN; 49; 1; 1994; 27-31) and 552 mg (3.1 mmol) isonicotinoyl chloride hydrochloride in 3 mL pyridine was heated at 120° C. over night. The reaction mixture was cooled and diluted with K₂CO₃ (sat) and extracted with three times 10 mL chloroform. The combined organic extracts were dried and concentrated. Flash chromatography (CH₂Cl₂/MeOH 10:1) afforded 83 mg (18%) of a white solid.

¹H NMR (300 MHz, CDCl₃): δ 8.65 (m, 2H), 7.67 (m, 2H), 4.13 (m, 2H), 3.24 (m, 2H), 1.91 (m, 2H).

In a similar manner following compound was synthesized:

| | | | |
|---|---|---|---|
| 9.2 | 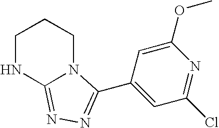 | 3-(2-Chloro-6-methoxy-pyridin-4-yl)-5,6,7,8-tetrahydro-[1,2,4]triazolo[4,3-a]pyrimidine | 36.5%400 mgWhiteSolid |

| | |
|---|---|
| ¹H NMR | (300 MHz, CDCl₃): δ 7.34(s, 1H), 6.93(s, 1H), 5.60(br, 1H), 4.112(t, |
| | 2H), 3.98(s, 3H), 3.52(m, 2H), 2.15(m, 2H) |

### Example 10

### 3-(2-Methoxy-pyridin-4-yl)-5,6,7,8-tetrahydro-[1,2,4]triazolo[4,3-a]pyrimidine

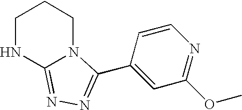

The title compound of Example 9.2 (200 mg) and the palladium on carbon catalyst 10% (100 mg) were combined. The reaction was then flushed with hydrogen gas. EtOH (3.2 mL) and triethylamine (0.6 mL) were also added to the vial. The solution was stirred over night at room temperature. The solution was then filtered through celite. A 10% 1 M NH₃ (in MeOH) in CH₂Cl₂ silica flash column was run in order to remove any traces of salt. The solution was concentrated to give the title product of Example 9 as a white solid powder (163 mg, 75% yield).

¹H NMR (300 MHz, CDCl₃): δ 8.27 (d, 1H), 7.28 (m, 1H), 6.99 (s, 1H), 6.05 (br, 1H), 4.14 (t, 2H), 4.1 (s, 3H), 3.6 (t, 2H), 2.1 (m, 2H)

### Example 11

### General Procedures for N-Alkylation of Isoxazole Sulphonates and Sulphonyl Chlorides

The isoxazole mesylate or chloride was weighted out into a vial and dimethylformamide (3 mL 1 mmol) was added to the solid. The vial was then flushed with argon. In a separate vial the aminotriazole (1.0 equivalents) was weighed and dissolved in tetrahydrofuran (6 mL/mmol). To this vial sodium tert butoxide (1.05 equivalents) or NaH was added and the vial was heated to 80° C. The contents of the mesylate containing vial was then added to the heated vial and the reaction was stirred for 3-30 minutes. An aqueous workup was then performed using EtOAc, water and Brine. The organic layers were then run through an Ex-Tube and concentrated in vacuo. A 10 g SPE columns were then used to purify the various products formed. The following table represents the couplings and the reaction conditions specific to each product.

The following compounds were synthesized as described above:

| 11.1 | 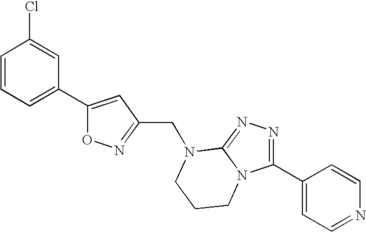 | 8-{[5-(3-Chlorophenyl)isoxazol-3-yl]methyl}-3-pyridin-4-yl-5,6,7,8-tetrahydro[1,2,4]triazolo[4,3-a]pyrimidine | 42%Whitesolid |
|---|---|---|---|
| ¹H NMR | (300 MHz, CDCl₃): δ 8.74(d, 2H), 7.76(s, 1H), 7.64(m, 3H), 7.39(m, 2H), 6.74(s, 1H), | | |
| | 4.88(s, 2H), 4.13(t, 2H), 3.44(t, 2H), 2.2(m, 2H) | | |
| 11.2 | 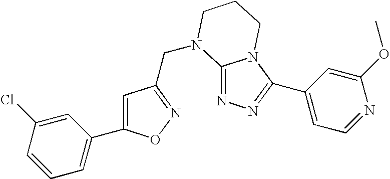 | 8-{[5-(3-Chlorophenyl)isoxazol-3-yl]methyl}-3-(2-methoxypyridin-4-yl)-5,6,7,8-tetrahydro[1,2,4]triazolo[4,3-a]pyrimidine | 42%Whitesolid |
| ¹H NMR | (300 MHz, CDCl₃): δ 8.27(d, 1H), 8.06(s, 1H), 7.96(d, 1H), 7.72(d, 1H), 7.6 (t, 1H), | | |
| | 7.3(s, 1H), 6.99(s, 1H), 6.83(s, 1H), 4.88(s, 2H), 4.1(t, 2H), 3.99(s, 3H), 3.44(t, | | |
| | 2H), 2.18(m, 2H) | | |
| 11.3 | 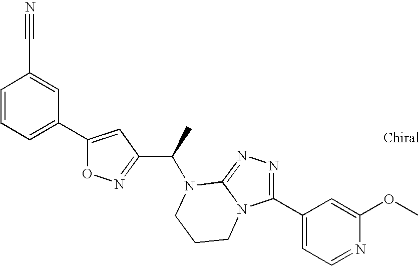 | 3-(3-{(R)-Methyl[3-(2-methoxypyridin-4-yl)-6,7-dihydro[1,2,4]triazolo[4,3-a]pyrimidin-8(5H)-yl]methyl}isoxazol-5-yl)benzonitrile | 67%(1.14 g)Foamingsolid |

| | |
|---|---|
| ¹H NMR | (300 MHz, CDCl₃): δ 8.28(d, 1H), 8(m, 2H), 7.71(d, 1H), 7.6(t, 1H), 7.3(s, 1H), 6.99(s, |
| | 1H), 6.71(s, 1H), 5.86(q, 1H), 4.08(m, 2H), 3.99(s, 3H), 3.42 (m, 1H), 3.28(m, |
| | 1H), 2.13(m, 2H), 1.78(d, 3H) |

The title chiral compound of Example 11.3 was obtained from the corresponding racemic compound by separation using Chiralpak AS with methanol at 1.0 mL/min flow rate (Rt=6.49 min).

### Biological Evaluation

### Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al, Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

### FLIPR Assay

Cells expressing human mGluR5d as described in WO97/05252 are seeded at a density of 100,000 cells per well on collagen coated clear bottom 96-well plates with black sides and experiments are done 24 h following seeding. All assays are done in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). Cell cultures in the 96-well plates are loaded for 60 minutes in the above mentioned buffer containing 4 μM of the acetoxymethyl ester form of the fluorescent calcium indicator fluo-3 (Molecular Probes, Eugene, Oreg.) in 0.01% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.800 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 90 second interval separates the antagonist and agonist additions. The fluorescence signal is sampled 50 times at 1 second intervals followed by 3 samples at 5 second intervals immediately after each of the two additions. Responses are measured as the difference between the peak height of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃).

GHEK stably expressing the human mGluR5d are seeded onto 24 well poly-L-lysine coated plates at 40×10⁴ cells/well in media containing 1 μCi/well [3H]myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with I unit/ml glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 ml perchloric acid (5%) on ice, with incubation at 4° C. for at least 30 min. Samples are collected in 15 ml polyproplylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 ml 30 mM ammonium formate. Next, total inositol phosphates is eluted with 8 ml 700 mM ammonium formate/100 mM formic acid and collected in scintillation vials. This eluate is then mixed with 8 ml of scintillant and [3H] inositol incorporation is determined by scintillation counting. The dpm counts from the duplicate samples are plotted and IC₅₀ determinations are generated using a linear least squares fitting program.

### Abbreviations

BSA Bovine Serum Albumin
CCD Charge Coupled Device
CRC Concentration Response Curve
DHPG 3,5-dihydroxyphenylglycine
DPM Disintegrations per Minute
EDTA Ethylene Diamine Tetraacetic Acid
FLIPR Fluorometric Imaging Plate reader
GHEK GLAST-containing Human Embrionic Kidney
GLAST glutamate/aspartate transporter
HEPES 4-(2-hydroxyethyl)-1-piperazineethanesulfonic acid (buffer)
IP₃ inositol triphosphate

Generally, the compounds were active in the assay above with IC₅₀ values less than 10 000 nM. In one aspect of the invention, the IC₅₀ value is less than 1000 nM. In a further aspect of the invention, the IC₅₀ value is less than 100 nM.

### Determination of Brain to Plasma Ratio in Rat

Brain to plasma ratios are estimated in female Sprague Dawley rats. The compound is dissolved in water or another appropriate vehicle. For determination of brain to plasma ratio the compound is administrated as a subcutaneous, or an intravenous bolus injection, or an intravenous infusion, or an oral administration. At a predetermined time point after the administration a blood sample is taken with cardiac puncture. The rat is terminated by cutting the heart open, and the brain is immediately retained. The blood samples are collected in heparinized tubes and centrifuged within 30 minutes, in order to separate the plasma from the blood cells. The plasma is transferred to 96-well plates and stored at −20° C. until analysis. The brains are divided in half, and each half is placed in a pre-tarred tube and stored at −20° C. until analysis. Prior to the analysis, the brain samples are thawed and 3 ml/g brain tissue of distilled water is added to the tubes. The brain samples are sonicated in an ice bath until the samples are homogenized. Both brain and plasma samples are precipitated with acetonitrile. After centrifugation, the supernatant is diluted with 0.2% formic acid. Analysis is performed on a short reversed-phase HPLC column with rapid gradient elution and MSMS detection using a triple quadrupole instrument with electrospray ionisation and Selected Reaction Monitoring (SRM) acquisition. Liquid-liquid extraction may be used as an alternative sample clean-up. The samples are extracted, by shaking, to an organic solvent after addition of a suitable buffer. An aliquot of the organic layer is transferred to a new vial and evaporated to dryness under a stream of nitrogen. After reconstitution of the residuals the samples are ready for injection onto the HPLC column.

Generally, the compounds according to the present invention are peripherally restricted with a drug in brain over drug in plasma ratio in the rat of <0.5. In one embodiment, the ratio is less than 0.15.

### Determination of In Vitro Stability

Rat liver microsomes are prepared from Sprague-Dawley rats liver samples. Human liver microsomes are either prepared from human liver samples or acquired from BD Gentest. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding a 3.5 times volume of acetonitrile. The concentration of compound remaining in each of the collected samples is determined by means of LC-MS. The elimination rate constant (k) of the mGluR5 inhibitor is calculated as the slope of the plot of In[mGluR5 inhibitor] against incubation time (minutes). The elimination rate constant is then used to calculate the half-life (T ½) of the mGluR5 inhibitor, which is subsequently used to calculate the intrinsic clearance (CLint) of the mGluR5 inhibitor in liver microsomes as: CLint.=(ln2× incubation volume)/(T ½× protein concentration)=μl/min/mg

### Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

### Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 ml/kg) in a foreleg vein. Ten min after i.v. administration, a nutrient meal (10% peptone, 5% D-glucose, 5% Intralipid, pH 3.0) is infused into the stomach through the central lumen of the assembly at 100 ml/min to a final volume of 30 ml/kg. The infusion of the nutrient meal is followed by air infusion at a rate of 500 mL/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal≦2 s before its onset in which case the relaxation is classified as swallow-induced. The pressure difference between the LES and the stomach should be less than 2 mmHg, and the duration of the complete relaxation longer than 1 s.

### Specimen Results are Shown in the Following Table:

| | FLIPR | Brain to Plasma |
|---|---|---|
| Example | mGluR5d (nM) | Ratio of compound in Rat |
| 11.2 | 51 | 0.36 |
| 11.3 | 40 | 0.09 |

1. A compound of formula (I) 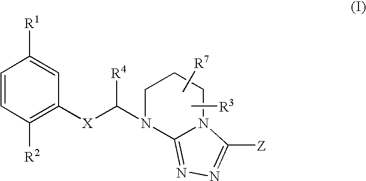
wherein
R¹ is methyl, halogen or cyano;
R² is hydrogen or fluoro;
R³ is hydrogen, fluoro or C₁-C₃ alkyl;
R⁴ is hydrogen or C₁-C₃ alkyl;
X is 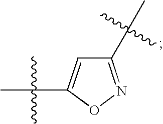
and Z is 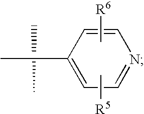
R⁵ is hydrogen, C₁-C₃ alkyl, C₁-C₃ haloalkyl, C₁-C₃ alkoxy; or C₁-C₃ haloalkoxy;
R⁶ is hydrogen, C₁-C₃ alkyl, C₁-C₃ haloalkyl, or C₁-C₃ haloalkoxy;
R⁷ is hydrogen, fluoro or C₁-C₃ alkyl;
as well as pharmaceutically acceptable salts, hydrates, isoforms, tautomers and/or enantiomers thereof.

2. A compound according to claim 1, wherein R¹ is halogen or cyano.

3. A compound according to claim 2, wherein R¹ is chloro.

4. A compound according to claim 2, wherein R¹ is cyano.

5. A compound according to claim 1, wherein R² is hydrogen.

6. A compound according to claim 1, wherein R³ is hydrogen or fluoro.

7. A compound according to claim 1, wherein R⁴ is hydrogen or methyl.

8. A compound according to claim 1, wherein R⁵ is hydrogen, C₁-C₂ alkyl or C₁-C₂ alkoxy.

9. A compound according to claim 1, wherein R⁶ is hydrogen, C₁-C₂ alkyl or C₁-C₂ alkoxy.

10. A compound according to claim 1, wherein R⁷ is C₁-C₂ alkyl or C₁-C₂ alkoxy.

11. A compound selected from
8-{[5-(3-Chlorophenyl)isoxazol-3-yl]methyl}-3-pyridin-4-yl-5,6,7,8-tetrahydro[1,2,4]triazolo[4,3-a]pyrimidine;
8-{[5-(3-Chlorophenyl)isoxazol-3-yl]methyl}-3-(2-methoxypyridin-4-yl)-5,6,7,8-tetrahydro[1,2,4]triazolo[4,3-a]pyrimidine; and
3-(3-{(R)-Methyl[3-(2-methoxypyridin-4-yl)-6,7-dihydro[1,2,4]triazolo[4,3-a]pyrimidin-8(5H)-yl]methyl} isoxazol-5-yl)benzonitrile
as well as pharmaceutically acceptable salts, hydrates, isoforms, tautomers and/or enantiomers thereof.

12. A compound according to claim 1 for use in therapy.

13. A pharmaceutical composition comprising a compound according to claim 1 as an active ingredient, together with a pharmacologically and pharmaceutically acceptable carrier.

14. Use of a compound according to claim 1, or a pharmaceutically acceptable salt or an optical isomer thereof, for the manufacture of a medicament for the inhibition of transient lower esophageal sphincter relaxations.

15. Use of a compound according to claim 1, or a pharmaceutically acceptable salt or an optical isomer thereof, for the manufacture of a medicament for treatment or prevention of gastroesophageal reflux disease.

16. Use of a compound according to claim 1, or a pharmaceutically acceptable salt or an optical isomer thereof, for the manufacture of a medicament for treatment or prevention of pain.

17. Use of a compound according to claim 1, or a pharmaceutically acceptable salt or an optical isomer thereof, for the manufacture of a medicament for treatment or prevention of anxiety.

18. Use of a compound according to claim 1, or a pharmaceutically acceptable salt or an optical isomer thereof, for the manufacture of a medicament for treatment or prevention of irritable bowel syndrome (IBS).

19. A method for the inhibition of transient lower esophageal sphincter relaxations whereby an effective amount of a compound according to claim 1 is administered to a subject in need of such inhibition.

20. A method for the treatment or prevention of gastroesophageal reflux disease, whereby an effective amount of a compound according to claim 1 is administered to a subject in need of such treatment or prevention.

21. A method for the treatment or prevention of pain, whereby an effective amount of a compound according to claim 1 is administered to a subject in need of such treatment or prevention.

22. A method for the treatment or prevention of anxiety, whereby an effective amount of a compound according to claim 1 is administered to a subject in need of such treatment or prevention.

23. A method for the treatment or prevention of irritable bowel syndrome (IBS), whereby an effective amount of a compound according to claim 1 is administered to a subject in need of such treatment or prevention.

24. A combination comprising (i) at least one compound according to claim 1 and (ii) at least one acid secretion inhibiting agent.

25. A combination according to claim 24 wherein the acid secretion inhibiting agent is selected from cimetidine, ranitidine, omeprazole, esomeprazole, lansoprazole, pantoprazole, rabeprazole or leminoprazole.

26. A compound selected from
3-[3-(1-hydroxyethyl)isoxazol-5-yl]benzonitrile;
1-[5-(3-Iodo-phenyl)-isoxazol-3-yl]-ethanol;
3-[1-(tert-Butyl-dimethyl-silanyloxy)-ethyl]-5-(3-iodo-phenyl)-isoxazole;
3-{3-[1-(tert-Butyl-dimethyl-silanyloxy)-ethyl]-isoxazol-5-yl}-benzonitrile; and
1-[5-(3-Cyanophenyl)isoxazol-3-yl]ethyl methanesulfonate.
